(19) United States
(12) Patent Application Publication
(10) Pub. No.: US 2003/0229379 A1
(43) Pub. Date: Dec. 11, 2003

(54) Method for cardioversion or defibrillation using electrical impulses at pacing strength

(76) Inventor: Maynard Ramsey III, Tampa, FL (US)

Correspondence Address: SEYFARTH SHAW, 55 EAST MONROE STREET, SUITE 4200, CHICAGO, IL, 60603-5803, US

(21) Appl. No.: 10/164,498

(22) Filed: Jun. 6, 2002

(51) Int. Cl. A61N 1/39

(52) U.S. Cl. 607/5

(57) ABSTRACT

A method for cardioverting or defibrillating a patient comprises the steps of: positioning a plurality of electrodes on a plurality of locations of the patient; and applying cyclically repeated steps of rapid sequenced stimulation at various cycle lengths, using generally pacing-strength electric currents sequentially delivered in rapid succession to the electrodes.

### BACKGROUND OF THE INVENTION

Prior art cardioversion and defibrillation methods typically require high energy shocks which may be painful and traumatic to the patient. A typical transthoracic cardioversion shock may have an energy range of between 80 joules and 200 joules. A typical transthoracic defibrillation shock may have an energy range between 100 joules and 400 joules. Both shocks are narrowly derived from an electrical pulse, either monophasic or multiphasic, having a duration between about 2 milliseconds and 10 milliseconds. Ordinarily, such shocks require that a conscious patient be anesthetized. Intracardial defibrillation shocks currently use an energy on the order of 10 to 50 joules. Cardioversion is basically the process of reducing the number of fibrillating, wild electrical wavelets that disorder the beating of the heart.

In Ramsey U.S. Pat. No. 5,928,270, a new method of cardioversion or defibrillation of a patient's heart is described in which different sets of a plurality of electrodes are pulsed with a plurality of pulses, within a maximum time period of 200 milliseconds. The use of multiple pulses over a maximum 200 millisecond time interval may be accomplished in which each pulse typically has an energy of between 0.04 joules and 40 joules, which technique is far less traumatic to the patient because the individual pulses each have less energy. As stated in the patent, the maximum duration of 200 milliseconds for such defibrillating is for the purpose of avoiding the t-wave, thus reducing the risk of setting off fibrillation of the heart. The energy may be spread out over a broader geometry than in the other prior art, using more electrodes, for less trauma to the patient, virtually to the point where the process is quite non-traumatic, while achieving desired cardioversion or defibrillation.

### DESCRIPTION OF THE INVENTION

By this invention, a method is provided for cardioverting or defibrillating a patient comprising the steps of: positioning a plurality of electrodes on a plurality of different locations of the patient and applying cyclically repeated sets of rapid-sequence stimulation, typically at various cycle lengths, using generally pacing-strength electric currents sequentially delivered in rapid succession to the electrodes. Typically, this process continues for a period of time substantially in excess of 200 milliseconds, with the energy of each pulse being typically no more than 600 millijoules and preferably no more than 10 millijoules. The rate of total pulses emitted from each of the plurality of electrodes may preferably be from 30 to 1,200 per minute, with the electrodes preferably emitting their pulses sequentially in a repeating cycle. Preferably, the electrodes emit their pulses sequentially at a pulse emission length of 1-50 milliseconds for each electrode.

This process comprising the pulsing of different electrodes preferably takes place at a pulse rate that substantially matches the natural cycle length of fibrillation of the heart of the patient, or at least that portion of the myocardium which is being stimulated by each set of electrodes. Cycle length determination is a known process, being typically and conventionally done by visual estimation by looking at electrograms, with the pulse cycle length being varied to find a cycle length that best captures an area of the heart, to take over pacing control of that area. However, the process can also be done automatically by computer.

Typically, the cardiac stimulation process of this invention continues for an indefinite length of time until cardioversion or defibrillation is achieved, or some arbitrary cutoff time is reached in which it becomes clear that the process is not successful, for example on the order of two to four minutes.

Fibrillation comprises a process in the heart where poorly organized or non-organized fibrillation waves are generated in a portion of the myocardium. The purpose of this invention in one circumstance is to cardiovert the heart by extinguishing these multiple fibrillation waves by local capture of large areas of the myocardium as a result of and in proximity to the sequentially and repetitively stimulated electrodes, preferably at a cycle length near the underlying local myocardial fibrillation cycle length. This repetitive stimulation is, as said before, at energy levels that typically approximate only that required for pacing of the heart rather than the significantly higher energy levels which have been up until now associated with and required for cardioversion and defibrillation. Often, the pulse energy may be on the order of one millijoule or less, for the patient's comfort.

For example, in the case of addressing atrial fibrillation, one or more sets of electrodes may be used. If plural, the respective electrode sets may be sequentially stimulated at the same or different cycle lengths. Specifically, one way to perform the method of this invention is to use a plurality of electrodes carried by a first catheter which is placed in the right atrium of the patient's heart, and another plurality of electrodes on a second catheter which is placed in the patient's esophagus, or the left atrium of the heart. These plural electrodes on each catheter can be sequentially activated to emit the desired pulses on a continuing basis until desired results are seen. Typically, the energy of each pulse may be no more than about 500 microjoules (0.0005 joule).

It may be desirable for the stimulating device which creates and controls the pulses to automatically determine the cycle length of the myocardium close to each set of electrodes and to display the current fibrillation cycle length on a screen or the like, to allow the operator to set the stimulation cycle length at a value to best capture large areas of the local area of the myocardium. Furthermore, the stimulating device may automatically determine the varying fibrillation cycle length of the myocardium close to each set of electrodes, and automatically set the stimulation cycle length of each set of electrodes to within about plus or minus 20 percent of the locally detected fibrillation cycle length.

Cardioversion is attempted by automatically stimulating at a frequency near the cycle length, followed by continuously modifying each stimulation cycle length such that it gradually more nearly approximates the natural cycle length which was previously determined for each electrode set. Typically, this process may begin when the varying fibrillation cycle length is near a maximum, a time known to be best for defibrillation, although it may be initiated at any time.

Also, a detector may be used which can determine when cardioversion has taken place. When this is achieved, the stimulating pulse device can automatically cease stimulation, or can substantially reduce the frequency of the stimulation typically in steps, to more closely approximate the rate of a normal rhythm. This process of post cardioversion pacing stimulation can prevent reinduction of fibrillation, flutter, or other tachyarrythmia. This detection of cardioversion may be achieved by monitoring the cardiac response during intervals between each sequence of stimulations of each set of electrodes. Alternatively, such a determination may be made by short interruptions in the rapid sequence of pulses at pacing energy, during which time of short interruption the heart may be monitored by one or more electrodes or other means, for the achievement of cardioversion.

One may also attempt cardioversion by the method of this invention and, if unsuccessful, one may deliver a sequence of larger energy defibrillation shocks, automatically, if desired, typically without prior cessation of local capture pacing, thus reducing the defibrillation threshold by pre-capturing large areas of myocardium at lower pacing energies typically of no more than about one to six millijoules, before delivering more defibrillation shocks. The larger defibrillation shocks may be single shock and single vector (i.e., in one direction from electrode to another electrode) or multi-shock, multi-vector (that is: using many electrodes and shock impulses going in various directions between various electrodes either simultaneously or sequentially). The electrodes used may be the same as are used for attempted sequential pacing cardioversion, or other cardiac, esophageal, or surface electrodes may be used alone in this process, or in combination with the pacing electrodes to apply such defibrillation shocks.

Thus, a method is provided in which cardioversion or defibrillation may be applied to the patient in a manner which greatly reduces or even eliminates the trauma, both cardiac trauma and the patient discomfort of conventional defibrillation and cardioversion requiring 1-400 Joules of energy.

This new method of pacing is a method of rapid sequence, multi-vector pacing, which permits painless cardioversion of atrial fibrillation and atrial flutter. The method may use esophageal electrodes, intracardiac electrodes, epicardial electrodes, and/or skin surface electrodes. If desired, automated stimulation protocols of the various electrodes in multi-vector manner may either be computer-generated or manual, based on the real-time determination of wavelets of cardiac excitation associated with fibrillation or flutter of the heart and the orientation and cycle length of said wavelets. If desired, pre-defined stimulation protocols or scripts may be provided by an electrophysiologist, and stored within memory of an automated apparatus for providing the rapid sequence, multi-vector pacing described in this application, for execution on command. Such a script may begin stimulation immediately, or it may wait until an advantageous moment to begin stimulation.

The CardioCommander electronic unit, available from CardioCommand, Inc. of Tampa, Fla. may be used for this process. As such rapid sequence, multi-vector pacing is being executed, the cardiologist may observe the recorded wave forms directly picked up from the heart, to determine if cardioversion is occurring or has occurred. If not, the cardiologist will in real time modify the energy, the rate, the sequence of the pulses, and/or their multi-vector pattern, to further attempt to cardiovert the heart by low energy, sequential pacing of multiple electrodes. These patterns of pulses may rapidly and sequentially stimulate the left atrium, for example, from electrodes in the esophagus, electrodes in the heart, and/or electrodes on the body surface, by using different combinations of electrodes to emit the desired pulses along different vectors between different electrodes, and at pacing energies as used in this invention. The rapid sequence, multi-vector pacing in accordance with this invention preferably attempts cardioversion by delivering a set of sequenced, multi-vector stimulations rapidly, repeating many times if necessary (for example, 2 to 500 times) until the area of local myocardial capture on the heart by the emitted pacing pulses creates enough conduction block on the heart to extinguish the arrhythmia. Since strong pacing currents are used, and not shocks of much higher energy, the patient may actually feel nothing, or very little as compared with the shock treatments of the prior art. Particularly, in some instances the energy of each pulse may be less than one half of a millijoule, although in other instances higher energies may be needed to locally capture the myocardium at each electrode location and create block to the fibrillation process.

This rapid, sequential, multi-vector pacing (preferably, repetitive stimulation at rates between 30-40 per minute and 1,200 per minute) also uses much less electrical current than prior art defibrillation processes. These lesser currents are generally not painful to the patient. They comprise multiple current vectors which are nearly simultaneous (separated in time by intervals on the order of one to ten milliseconds). These current vectors, as stated, create multiple areas of conduction block in the heart by regional entrainment or local capture. Thus, the wild currents of fibrillation running across the heart are blocked, and forced into a coherent pattern again by the organized pulses provided to the heart by the sequential pacing of multiple electrodes in accordance with this invention.

If desired, biphasic or other multi-phasic waveforms may be used in the practice of this invention.

If desired, the multiple, sequentially-created current vectors used in this invention may be created by using electrodes exclusively within the esophagus, or in the heart, or on the heart's surface. Alternatively, these various electrodes may be used in combination to create multiple, different current vectors by the rapid, sequential utilization of different patterns of electrodes. Current vectors from the esophagus to the chest surface, or to electrodes within the right atrium, and/or the left atrium of the heart, may allow pacing and capture of portions of the atrial septum as well as the posterior left atrium, to provide conduction block of the wavelets which perpetuate the arrhythmia on the posterior left atrium and parts of the septum and right atrium.

The use of multiple current vectors with rapid, sequential stimulation of the electrodes generally at pacing strengths has the following advantages: (1) patient discomfort may be reduced by the smaller current strengths and (2) “vector shorting” may be avoided, which is a circumstance that can occur if all electrode vectors were stimulated at once. By the technique of this invention, a large zone of local capture of myocardial tissue may be achieved, so that the heartbeat begins to obey the patterns artificially imposed by the electrodes used. This also results in a conduction block, typically across the posterior left atrium when paced from the esophagus, or from the right atrium or left atrium, to temporarily and non-invasively partition portions of the heart with one or more regions of conduction block, to extinguish one or more circulating fibrillation wavelets, and also to block the progression of wild wavelets of fibrillation which have not been extinguished.

### DESCRIPTION OF DRAWINGS

In the drawings, FIG. 1 is a diagrammatic view of a human heart, showing two catheters inserted into the body of the patient, each catheter bearing multiple electrodes.

FIG. 2 is a chart showing the amplitude and times of groups of stimulating pulses applied to a heart.

### DESCRIPTION OF SPECIFIC EMBODIMENTS

Referring to the drawings, a human heart 10 is schematically shown, with the right atrium RA and the left atrium LA being respectively illustrated. A catheter 12 is shown to be inserted by conventional technique into the right atrium, the catheter carrying ten electrodes 14a-j, each of said electrodes being separately connected to a conductor wire that extends out of the body and to an electronic control unit 16, to permit independent electrical pulses with each electrode as controlled by unit 16. A second catheter 18 is shown to be positioned in the esophagus adjacent to the heart and not in it, although, alternatively, catheter 18 may also extend in conventional manner into the left atrium, or right atrium, if desired. Catheter 18 carries six independent electrodes with separate conductors which extend through the catheter and from the body into communication with control unit 16. Other electrodes 20, 22 may, as desired, be placed on the skin of the patient, having conductors 24, that communicate with control unit 16.

The process of rapid sequence, multi-vector pacing, can proceed with this arrangement to cardiovert or defibrillate a patient by the following process:

A series of stimulating pulses of generally pacing strengths, in contrast to the much higher conventional cardioverting strength, may be utilized. This series of pulses may be created by sequentially activating circuits and providing a pulse of electrical current between (as shown by the arrows): first, electrodes 14a and 14b, followed by a pulse of current between electrodes 14c, 14d, followed by a pulse of current between electrodes 14e, 14f, and so on to the last pulse of current between electrodes 14i, 14j. This series of pulses is then repeated in similar, sequential manner again and again, being optionally modified in a manner that the cardiologist will choose, in an attempt to conform to approximately the cycle length of the fluttering or fibrillation in the right atrium or the left atrium for better capture. As a typical result, a portion of the heart tissue will become subject to the pacing signals emitted from catheter 12, to create a zone of local capture 24 in the heart tissue, causing a conduction block in the right atrium (as illustrated) of the wild and unorganized fibrillation wavelets. When maintained for a period of time such as several seconds or a minute, the pulses, in many circumstances, will cause the extinguishment of the undesirable fibrillation wavelets, bringing the heart under control of the pacing provided from control unit 16.

At the same time, or another time, the left atrium can be treated in similar manner, in which the various independent electrodes 20a-20f are similarly sequentially pulsed to provide brief electrical current pulses respectively first between electrodes 20a, 20b, and then electrodes 20b, 20c, followed by electrodes 20c, 20d; 20d, 20e; and finally 20e, 20f. Alternatively, in either of the above circumstances, the same electrode or other electrodes may be used to generate pulses having different vectors, for example, from electrode 20c to 20b in opposite direction between electrodes 20b, 20c, as part of the above described series of pulses. Basically, any current pulse may pass between any electrodes, as programmed by the cardiologist, in any direction, including electrodes in or on the heart, the body surface, or the esophagus. As this sequence of electrical pulses proceeds over a period of seconds, capture or control of another significant area 26 of the heart may be achieved, this area being positioned on the left atrium. As before, wild signals of flutter and fibrillation can be blocked by the captured zone 26, resulting in the extinguishing of the undesired wavelets.

As shown by the arrows connecting the respective electrodes 14a-j and 20a-f, this particular arrangement gives a total of ten stimuli, as shown by the curved arrows, for each complete set of stimulation cycles. The pulse delays, strengths, and wave shapes for each discrete stimulus are shown to all be the same for each set in this particular circumstance, but actually they may be varied as desired for greater effect and/or for greater patient comfort.

As a specific example, a complete sequence of six stimuli provided by the right atrial catheter 12 may be provided every 200 milliseconds, with a delay of one millisecond between each of the pulses. Each pulse may have a duration of two to eight milliseconds and an amplitude of two to 12 milliamps, being typically monophasic, biphasic or triphasic in shape as desired. The overall energy of each of these pulses may be approximately 0.0001 to 0.06 millijoule, depending in part on the resistance between the respective electrodes (assumed to be 10 to 50 ohms).

Turning to the stimulus sequence parameters on the esophageal catheter 18, as another specific example, a cycle length of 215 milliseconds may be used, with a delay between stimuli of three milliseconds, with the stimulus (pulse) strength and shape being monophasic, with 10 to 25 milliamps of amplitude, and a duration of ten milliseconds for each pulse. The energy of the pulses here maybe on the order of 0.5 to 3.125 millijoule, depending again on the resistance between the electrodes (assumed to be 500 ohms). Each cycle of stimulating pulses may be simultaneously or sequentially emitted from each catheter, repeating the series at the approximately 1/5 second intervals indicated above until the respective capture areas 24, 26 are formed, where the rhythm of the heartbeat is then controlled by the signals emitted from the catheters rather than from the fibrillation wavelets. Then, cardioversion or defibrillation can be achieved, since the number of wavelets has been reduced below a critical number by the local capture achieved by this stimulation.

Following such termination, the stimulating cycle may be terminated completely, or altered in a manner calculated to encourage the heart to return to a normal beat without flutter or fibrillation, by pacing the heart at above its intrinsic rate to help suppress reinitiation.

The signals of either catheter may be biphasic if desired. For example, the pulse may be created from a positive electrode 20a and a negative electrode 20b, and then immediately repeated as part of the same pulse with reverse polarity. In a triphasic signal, the polarity may shift three times in a single pulse.

As an alternative, or in addition to the above described signals, pulses may pass between respective electrodes of the different catheters. For example, a first pulse may pass between electrodes 20b and 14c, followed by a pulse between electrodes 20c and 14e, followed by a third pulse between electrodes 20d and 14g. As a specific example, the cycle of such pulses may repeat every 210 milliseconds (the “cycle length”), with a delay between the respective pulses of two milliseconds. The pulse shape may be monophasic, with an amplitude of 16 milliamps and a duration of six milliseconds, providing a total energy of each pulse on the order of 0.03 millijoule, depending on the resistance in the heart (assumed at 200 ohms). An area of local capture which extends between the two catheters 12, 18 can be expected.

As another option, a new series of multiple current vectors can be created by the use of conventional electrodes 22, 28 carried on the skin of the patient, interacting with the respective electrodes carried on the catheters 12, 18. Typically, skin electrodes 22, 28 may be one and the same electrode, although alternatively one may be placed for example on the front and one on the back of the patient, or left and right on the chest, to obtain different current vectors if desired. By way of specific example, pulses could be generated in sequential manner between the respective electrodes 20b, 20c, 20d, and 20e and skin electrode 22, as shown by current vector arrows 30. Similarly, pulses could be generated between electrodes 14c, 14e, and 14g and skin electrode 28 as shown by current vector arrows 32. These pulses could be fitted into the multiple vector series of pulses described above between the respective electrodes 14 on catheter 12 and the respective electrodes 20 on catheter 18, to provide yet another pattern of local capture resulting in cardioversion or defibrillation, which proceeds in accordance with the instructions of the cardiologist. This multiple vector pattern of greater complexity is typically repeated over and over in a continuing cycle until a sufficiently large area of heart tissue capture is achieved, and the results of cardioversion or defibrillation are established.

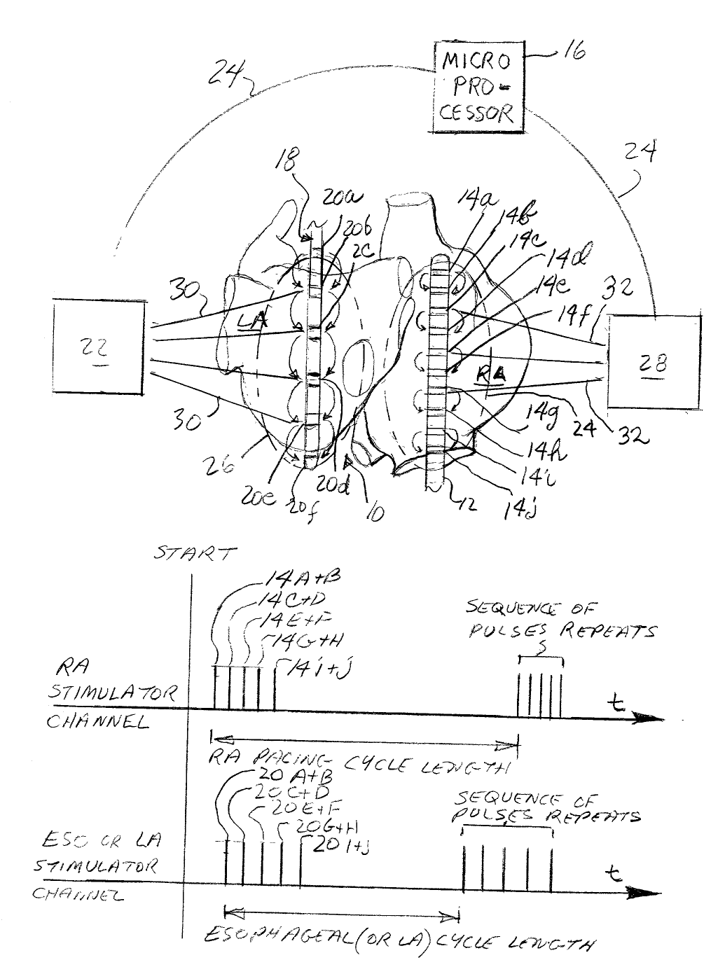

Referring to FIG. 2, examples of a particular pacing pattern which may be used for cardioversion with respect to the setup of FIG. 1 is shown.

Upper graph 40 shows two groups of an indefinitely long repeating sequence of electrical stimulating pulses, with graph 40 showing the energy of the pulses between specified electrodes on the vertical axis and the timing on the horizontal axis. This series of pulses of this embodiment is produced by the electrodes 14 carried on catheter 12, which occupies the right atrium. It can be seen that the first pulse is between electrodes 14a and b; the second pulse is between electrodes 14c and d; the third pulse is between electrodes 14e and f; the fourth pulse is between electrodes 14g and h; and the fifth pulse is between electrodes 14i and j. The quantitative amplitude and timing of these pulses may be as previously described, as well as the spacing between each of the groups of pulses. It can be seen that the spacing between the groups of pulses is substantially larger than the spacing between the individual pulses in the group.

Additional groups of pulses for electrodes 14 are created in similar manner, with similar timing, until either (1) definite results are observed, or (2) a conclusion may be drawn that the desired results are not going to be achieved, typically a period of time of a minute or two.

The lower graph 50 is similar with the vertical axis, representing the energy or amplitude of respective pulses in a pair of spaced groups, with the time of the pulses being shown on the horizontal axis. These pulses are emitted from electrodes 20 of catheter 18, which resides in the esophagus (or alternatively the left atrium). Specifically, in this embodiment, the first pulse of each group travels from electrode 20a to electrode 20b; the next pulse travels from electrode 20b to 20c; the third pulse travels from electrode 20c to 20d; the fourth pulse travels from electrode 20d to 20e; and the fifth pulse travels from electrode 20e to 20f. The following group of pulses is similar in amplitude and time spacing, with similar groups of pulses being repeated for an indefinite period until the desired results are obtained, or it becomes clear that a change in treatment is required.

Also, it can be seen in this embodiment that the space of time between each of the individual pulses is wider in the series of pulses of lower graph 50 than in the series of pulses of graph 40, indicating that the time between the respective pulses is larger in this case of graph 50. Specifically, the timing and the pulse amplitudes may be as previously described.

The various parameters of the cycle and the pulses may be adjusted individually in real time by appropriate control of the electronics 16, with the cardiologist attempting one mode or another mode of pulse patterns until maximum local capture of heart tissue is achieved in both the left atrium and the right atrium, and cardioversion occurs. Alternatively, only a single catheter may be used if desired, for example, the esophageal catheter 18, in an attempt to cardiovert or defibrillate without insertion of catheters into the heart. Alternatively catheters in the left atrium could be used in addition or in place of others. As a means of enhancing the chances of quick cardioversion using the rapid sequence multivector pacing method, a control unit could automatically modify stimulation parameters over a range of values until cardioversion is achieved.

The zones of local capture 24, 26 may be substantially larger than those which are achieved by conventional pacing of an electrode pair. The increased area of myocardial capture provides improved pacing and cardioversion in accordance with this invention.

The above has been offered for illustrative purposes only, and is not intended to limit the invention of this application, which is defined in the claims below.

That which is claimed is:

1. A method for cardioverting or defibrillating a patient, comprising the steps of: positioning a plurality of electrodes on a plurality of different locations of the patient; and
applying cyclically repeated sets of rapidly sequenced stimulation, using generally pacing-strength currents sequentially delivered in rapid succession to said electrodes.

2. A method as defined in claim 1, in which a cycle length used is generally comparable to the cycle length of the underlying fibrillating or fluttering myocardium area closest to the electrodes.

3. The method of claim 1 in which said stimulation is applied at varying cycle lengths.

4. A method as defined in claim 1, in which at each cycle of stimulation, multiple pairs or sets of multiple electrodes are stimulated individually and in rapid sequence on a repeating basis of at least one pulse from each electrode used every two seconds, with the energy of each pulse being no more than 600 millijoules; and continuing said pulsing until said cardioverting or defibrillating occurs.

5. The method of claim 4 in which the energy of each pulse is no more than ten millijoules.

6. The method of claim 4 in which said electrodes emit said pulses sequentially in a repeating cycle, in response to manual or automatic control.

7. The method of claim 4 in which said electrodes emit said pulses sequentially at a pulse emission rate of 1-50 milliseconds per pulse.

8. The method of claim 4 in which said pulsing of different electrodes takes place at a pulse rate that substantially matches the natural cycle length of fibrillation of at least a portion of the heart of the patient.

9. The method of claim 8 in which the fibrillation is atrial fibrillation.

10. The method of claim 4 which is performed using a plurality of electrodes carried by a first catheter placed in the right atrium of the patient's heart and a second catheter placed in the patient's esophagus.

11. The method of claim 4, which is performed using a plurality of electrodes carried by a first catheter placed in the right atrium of the patient's heart and a second catheter placed in the left atrium of the patient's heart.

12. The method of claim 4 in which the energy of each pulse is no more than ten millijoules.

13. The method of claim 4 in which the stimulating device automatically ceases stimulation or substantially reduces the frequency of stimulation, once it automatically detects that cardioversion has taken place.

14. The method of claim 4 in which said detection of cardioversion may be achieved by monitoring the cardiac response during intervals between sequences of stimulations of each set of electrodes, or such determination may be made by short interruptions in the rapid pacing during which short interruption the heart is monitored for the achievement of cardioversion.

15. The method of claim 4 in which a stimulating device automatically determines when cardioversion has occurred and automatically reduces the frequency of stimulation in steps to more closely approximate the rate of a normal rhythm to prevent reintroduction of fibrillation, flutter or other tachyarrythmia.

16. The method of claim 4 in which the device attempts cardioversion by said method and, if unsuccessful, delivers a sequence of larger defibrillation shocks automatically, thus reducing the defibrillation threshold by pre-capturing large areas of myocardium before delivering the defibrillation shocks.

17. The method of claim 16 in which the larger defibrillation shocks are single shock and single vector, or multi-shock, multi-vector, using the same electrodes as used for attempted cardioversion.

18. The method of claim 17 in which other cardiac, esophageal or surface electrodes alone, or in combination with pacing electrodes, are used to apply said larger defibrillation shocks.

19. The method of claim 4 in which a stimulating device automatically or manually begins pacing at a pre-determined, slower cycle length after the accomplishment and detection of cardioversion by the operator or automatically by the device, so as to prevent the reintroduction of fibrillation or flutter to the heart.

20. A method for cardioverting or defibrillating a patient, comprising the steps of: positioning of plurality of electrodes on a plurality of different locations of the patient;
applying cyclically repeated sets of rapidly sequenced stimulation, using generally pacing-strength currents sequentially delivered in rapid succession to said electrodes; and
detecting cardioversion by monitoring the cardiac response during intervals between sequences of stimulations of each set of electrodes or by short interruptions in the rapidly sequenced stimulation, during which short interruption the heart is monitored for the achievement of cardioversion.

21. The method of claim 20, in which, after achievement of cardioversion, the frequency of stimulation is automatically reduced to more closely approximate the rate of a normal rhythm to prevent reintroduction of fibrillation, flutter, or other tachyarrythmia.

22. The method of claim 20 in which the energy of each pulse is no more than 10 millijoules.

23. The method of claim 4, which is performed using a plurality of electrodes carried by a single catheter placed in the patient's esophagus.
